(19) United States
(12) Patent Application Publication
(10) Pub. No.: US 2022/0168454 A1
(43) Pub. Date: Jun. 2, 2022

(54) VAPORIZER OF DECONTAMINATING AGENTS

(71) Applicant: TEMA SINERGIE S.P.A., Faenza (IT)

(72) Inventor: Stefano PIANCASTELLI, Brisighella (IT)

(21) Appl. No.: 17/601,682

(22) PCT Filed: Apr. 2, 2020
(86) PCT No.: PCT/IB2020/053149

§ 371 Date: Oct. 5, 2021

(30) Foreign Application Priority Data
Apr. 8, 2019 (IT) 102019000005314

(51) Int. Cl. A61L 2/20; A61L 9/03; A61L 2/26

(52) U.S. Cl. CPC A61L 2/208; A61L 9/03; A61L 2/26; A61L 2209/211; A61L 2202/15; A61L 2202/11; A61L 2209/135; A61L 2202/14

(57) ABSTRACT

Vaporizer of decontaminating agents, preferably oxygen peroxide, comprising a vaporization chamber (1) with hot walls (5), a first inlet (2) in said chamber (1) of a flow of a vector fluid, preferably air, directed towards the hot wall (5), a second inlet (3) in said chamber (1) of a pressurized flow of a liquid decontaminating agent, interfering in a meeting point (P) with said flow of vector fluid to be dragged and vaporized in contact with the hot wall (5), an outlet (4) from the chamber (1) of a flow of vaporized decontaminating agent conveyed by said vector fluid, comprising injection means (6) associated with said second inlet branch (3) for injecting an atomized flow of micro-drops of decontaminating agent.

### TECHNICAL FIELD OF THE INVENTION

The invention relates to a vaporizer of decontaminating agents, and in particular to a hydrogen peroxide vaporizer for sterilization plants, for example of environments or containers for sanitary or industrial use.

### BACKGROUND ART

At present, devices are known on the market for the generation of hydrogen peroxide, in a highly concentrated vapour state, for decontaminating sealed chambers such as: Isolators, airlocks, preparation-passers or handling cells.

In fact, hydrogen peroxide (H2O2) is known for its broad-spectrum decontamination power and its use as a decontaminating agent is now a standard in various industries, especially the food and pharmaceutical industries. The purpose of these devices is therefore to generate an airflow loaded with hydrogen peroxide which can be conveyed to the environment to be decontaminated.

In particular, for example from EP1425048, vaporizers are known comprising a metal mass with high thermal capacity which provide an airflow branch of pressurized air and an injection branch of the peroxide which meet inside the vaporizer so that the peroxide is conveyed by the air on the hot walls, causing their vaporization before being extracted by an outlet nozzle and used as a decontaminating agent.

Generally the peroxide in liquid form is introduced into the airflow through suitable drippers, fed by peristaltic pumps, consequently with a fairly coarse control of both the diameter of the drop that must vaporize and the peroxide flow (g/min). US2004/237466 discloses a vaporizer in which an injection nozzle is used which is large enough to generate a solid/liquid jet with a diameter of a few tenths of a millimetre without the risk of clogging.

However, these systems have some drawbacks, in particular in relation to the efficiency of the vaporization with respect to the energy used and to the precision and control of the injected peroxide.

### OBJECT OF THE INVENTION

A first object of the invention is to propose a vaporizer free from the above-mentioned drawbacks which guarantees a high vaporization efficiency of the decontaminating agent introduced into the vaporizer.

A further object is to propose a vaporizer which guarantees high precision and repeatability of the step of introducing the decontaminating agent into the vaporizer.

### SUMMARY OF THE INVENTION

These and further technical aims and advantages have been achieved according to the invention with a device according to at least one of the attached claims.

A first advantage obtained according to the invention consists essentially in the fact that the use of an injection nozzle with a calibrated diameter allows, together with the pump that generates upstream pressure, injecting atomized peroxide in the form of micro-drops, which compared to the injection of non-atomized drops with the same quantity of peroxide, increases the energy exchange surface with the hot walls of the vaporization chamber.

A second advantage consists in the greater precision in the dosage of decontaminating agent introduced into the vaporizer thanks to the use of a volumetric pump combined with a step motor with encoder, which guarantees high repeatability and precision thanks to the direct feedback of the integrated encoder.

A further advantage consists in the cyclonic form of the vaporization chamber, which by forcing the air to make spiral trajectories pushes the micro-drops of peroxide onto the hot walls of the vaporizer by centrifugal force, guaranteeing the immediate and complete vaporization of the injected liquid.

The cyclonic shape also allows the vaporization chamber to be realized in a thin wall, i.e. with a high ratio between the volume of the vaporization chamber and the total volume (chamber volume+heated mass), all while maintaining geometries that couple well with easily available heating systems. The thin wall of the vaporizer considerably lowers its thermal capacity, allowing a much more precise control of the vaporization temperature and an optimization of the power used for vaporization with the same peroxide flow.

### LIST OF DRAWINGS

These and other advantages will be better understood by anyone skilled in the art from the description below and the accompanying drawings, given as a non-limiting example, wherein:

FIG. 1 shows a vaporizer according to the invention;

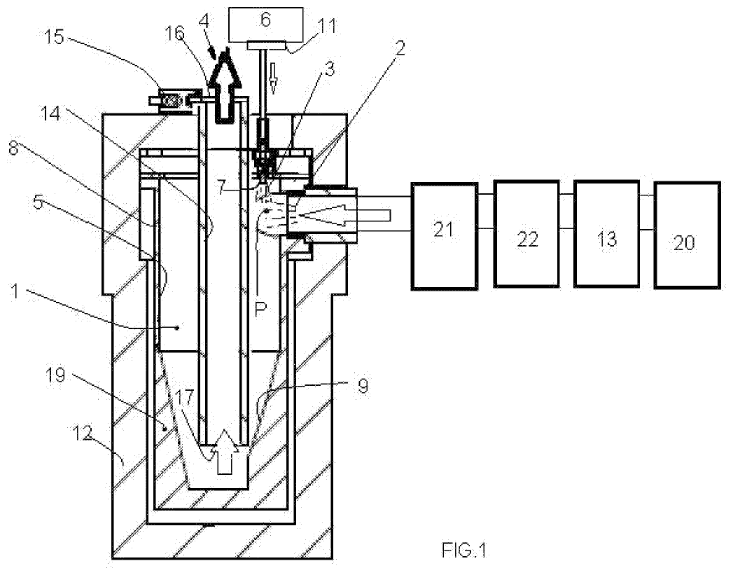

FIGS. 2, 2a, 2b, 2c respectively show a side view, in the section A-A, from above, and a perspective view of a vaporization chamber according to the invention.

### DETAILED DESCRIPTION

With reference to the attached drawings, a vaporizer of decontaminating agents, preferably oxygen peroxide, is described.

The vaporizer comprising a vaporization chamber 1 with hot walls 5 with external insulation 12, into which a first inlet 2 of a flow of a vector fluid, preferably dry, hot air driven by a fan 13 with a controlled, dehumidified, filtered and heated flow rate by suitable means 20, 22, 21 arranged upstream of the inlet 2 into the chamber 1.

By way of example, the preheating means 21 can be constituted by suitable resistances inserted inside the air ducts; the dehumidification means, by way of example, can be via a refrigeration cycle or a silica gel, the latter with rotor or static technologies.

Preferably, the temperature of the air entering the chamber 1 is about 55-65° C., controlled for example via thermocouple, while the temperature of the vapour flow leaving the vaporizer is about 70-80° C., measured for example by a thermocouple, but not necessarily controlled.

The flow of vector fluid is directed towards the hot wall 5 in a tangential direction thereto, and encounters inside the chamber 1 a flow of a liquid decontaminating agent, for example a 35% solution of oxygen peroxide, coming from a second inlet 3 and interfering with the flow of vector fluid at a meeting point P, for example being injected transversely thereto, to be dragged and vaporized via thermal flash in contact with the hot wall 5.

Preferably, the pressure in the injection inlet 3 of the decontaminating agent is controlled by means of a pressure switch located on the injection line, in order to avoid injecting into a clogged nozzle. By way of example, the pressure switch threshold can be set around 6 bar.

The chamber 1 further comprises an outlet 4 of the flow resulting from the meeting between the vaporized decontaminating agent and the transport vector fluid. According to the invention, the vaporizer comprises injection means 6 arranged upstream of the second inlet 3 and associated therewith to inject an atomized flow of micro-drops of decontaminating agent.

Preferably, the injection means comprise a pump 6, for example a volumetric pump, equipped with an encoder 11 to feed the second inlet 3 with a controlled flow of liquid decontaminating agent equal to about 5.0-50.0 g/m in, preferably 5.5-30.0 g/m in at a pressure between 1 and 4 bar, preferably 2-4 bar, established according to the injection rate of the decontaminating agent, a higher rate requiring a higher pressure at the injection nozzle 7.

In the various possible operating conditions, the nozzle 7 can be equipped with calibrated injection holes in the chamber 1 with an average diameter comprised between 0.05 and 0.25 mm, preferably 0.22 mm.

Equivalently, the peroxide injection circuit can consist of a pressurized tank with an ON/OFF dosing valve: the pressure of the pressurized tank containing liquid peroxide can be maintained by compressed air or, if containing only liquid, by means of an electronically controlled diaphragm pump or gear pump.

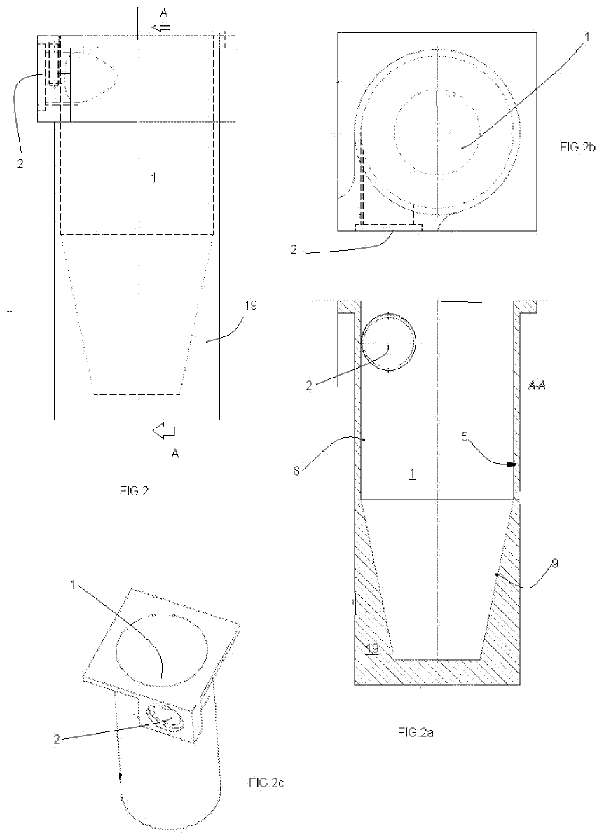

With reference in particular to FIGS. 2, 2a-2c, the vaporization chamber 1 is obtained in a hollow metal body 19 so as to obtain a ratio between the volume of the vaporization chamber and the total volume given by the sum of the volume of the chamber 1 and of the heated body 19 greater than 55%.

This feature allows obtaining high energy efficiency and reducing thermal inertia. Furthermore, the geometry of the chamber 1 is such as to present a ratio between the vaporization surface defined by the hot wall in contact with the decontaminating agent and the vacuum volume, i.e. the volume of the vaporization chamber greater than 0.4 cm−1.

This feature allows obtaining high productivity and reducing the quantity per surface unit of the residue that forms on the hot surface upon the vaporization by contact, and which tends to reduce the efficiency of the vaporizer and require cleaning and maintenance.

Preferably the chamber 1 has a cyclone geometry comprising a first portion 8 of a hot wall near the inlet 2 formed by a cylindrical section wall, for example made of metal, preferably aluminium, and a second conical portion 9 of hot wall near the outlet 4, converging in the direction of the vapour flow.

Advantageously, the tangential introduction of the airflow and the geometry of the vaporization chamber determine a spiral trajectory of the flow of atomized decontaminating agent which is transported by the vector fluid from the meeting point P towards the hot wall 5 and therefore, by centrifugal force, is kept in contact with the first portion 8, vaporized by thermal flash, and induced to the outlet 4 due to the convergent conical geometry of the second portion 9 of the chamber 5.

In the illustrated embodiment example, the outlet 4 is equipped with a temperature sensor 15 of the vapour produced and comprises a duct 14 arranged inside the chamber 1 and extended from an internal lower point 17 near the convergent end of the conical portion 9 to an external upper point of extraction of the vaporized decontaminating agent 16.

In an operating example, the airflow generated by the fan 13 is previously dehumidified by a dryer 20 and the air entering the chamber 1, possibly filtered by a filter 22, is pre-heated by a heating unit 21, so as to enter the vaporizer hot and dry and therefore ready to support vapour.

The hot, dry airflow is then conveyed into the vaporizer, where the meeting point P is located between the airflow and the injection flow, transversal to each other. When the step pump 6 pushes the solution of decontaminating agent, in the example described oxygen peroxide, towards the vaporizer, the small size of the nozzle 7 and the ability of the pump 6 to generate pressure result in an atomization of the liquid which therefore splits into micro-drops passing through the nozzle.

These micro-drops collide with the airflow in the vaporizer. The cyclonic shape of the vaporizer thus obliges the flow of vector fluid that enters tangent to the upper surface of the inside of the vaporizer and the micro-drops dragged by the vector fluid to follow spiral trajectories, while the centrifugal force pushes the micro-drops towards the hot walls of the vaporization chamber.

The result is a thermal flash that brings the peroxide solution to the vapour state. The airflow will thus be charged with peroxide vapour and will be conveyed and pushed by the prevalence of the fan to the bottom of the vaporizer, at the portion 9 of the hot wall, towards the outlet 4 and from there to the environment or the container by means of one or more diffuser nozzles.

The present invention has been described according to preferred embodiments, but equivalent variants can be conceived without departing from the scope of protection granted.

1. A vaporizer of decontaminating agents, the vaporizer comprising
a vaporization chamber with a hot wall,
a first inlet in said chamber of a flow of a vector fluid directed towards the hot wall,
a second inlet in said vaporization chamber of a pressurized flow of a liquid decontaminating agent, interfering in a meeting point with said flow of vector fluid to be dragged and vaporized in contact with the hot wall,
an outlet from the vaporization chamber of a flow of vaporized decontaminating agent transported by said vector fluid, and
injection means associated with said second inlet branch configured to inject an atomized flow of micro-drops of decontaminating agent.

2. The vaporizer according to claim 1, wherein said injection means comprise a pump for feeding a flow of liquid decontaminating agent at a pressure of between 1 and 4 bar and at least one nozzle with calibrated injection holes in the vaporization chamber with an average diameter comprised between 0.05 and 0.25 mm.

3. The vaporizer according to claim 2, wherein said injection means are configured to inject from 5.5 g/min to 30 g/min of liquid decontaminating agent at a pressure comprised between 2 and 4 bar increasing with the growth of said pressurized flow of liquid decontaminating agent.

4. The vaporizer according to claim 2, wherein said pump is a volumetric pump with encoder.

5. The vaporizer according to claim 1, wherein said inlet is configured to introduce the flow of vector fluid in a tangential direction to the hot wall and said vaporization chamber has a cyclone geometry with a first portion in a proximal position with respect to said first inlet and a second portion with a conical geometry converging in the flow direction in order to induce a spiral trajectory of said flow of vaporized decontaminating agent carried by said vector fluid between said meeting point and said outlet.

6. The vaporizer according to claim 1, wherein said vaporization chamber is obtained in a hollow metal body with a ratio between the volume of the vaporization chamber and the total volume given by the sum of the volume of the chamber and of the hollow metal body greater than 55%.

7. The vaporizer according to claim 1, the ratio between the vaporization surface defined by the hot wall in contact with the decontaminating agent and the volume of the vaporization chamber is greater than 0.4 cm−1.

8. The vaporizer according to claim 1, wherein said vaporization chamber is metallic.

9. The vaporizer according to claim 1, comprising means for pre-heating said flow of vector fluid upstream of said first inlet.

10. The vaporizer according to claim 1, wherein the outlet is equipped with a temperature sensor of the vaporized decontaminating agent produced.

11. The vaporizer according to claim 1, wherein the outlet comprises a duct arranged inside the vaporization chamber and extended from an internal inlet point to an external extraction point.

12. The vaporizer according to claim 1, comprising an external insulation of said vaporization chamber.
